(19) United States
(12) Patent Application Publication
(10) Pub. No.: US 2022/0025415 A1
(43) Pub. Date: Jan. 27, 2022

(54) Method for Producing y-aminobutyric Acid (GABA) by Fermentation of Native Strain from Raw Material of Sayram Ketteki

(71) Applicant: Tarim University & Technology, alar city (CN)

(72) Inventors: WeiHua Wang, alar city (CN); Dongla Gao, alar city (CN); Ruicheng Guo, alar city (CN); Xinyu Shen, alar city (CN); Yiteng Zhang, alar city (CN); Jing Liu, alar city (CN)

(21) Appl. No.: 17/380,714

(22) Filed: Jul. 20, 2021

(30) Foreign Application Priority Data
Jul. 24, 2020 (CN) 2020107223260

(51) Int. Cl. C12P 13/00; C12N 1/20

(52) U.S. Cl. CPC C12P 13/001; C12N 1/20; C12N 2500/30; C12N 2500/34; C12N 2500/84; C12N 2500/05

(57) ABSTRACT

The present invention discloses a method for producing γ-aminobutyric acid (GABA) by fermentation of native strain from raw material of Sayram Ketteki, comprising the following steps: dissolving the cow's milk in the normal saline for dilution to obtain diluents with different gradients first, coating the diluents in solid media for culture respectively, isolating single colonies and storing them in a glycerin cryopreservation tube for cryopreservation respectively; next, inoculating them in liquid medium for culture respectively, and inoculating them in fermentation medium for culture for culture after activation. The content of GABA product obtained by the present invention can reach 450 μg/mL.

### CROSS REFERENCE TO RELATED APPLICATION

The present invention refers to the technical field of γ-aminobutyric Acid (GAB A) extraction process, in particular to a method for producing γ-aminobutyric acid (GABA) by fermentation of native strain from raw material of Sayram Ketteki.

### BACKGROUND OF THE INVENTION

GABA is a natural non-protein free amino acid, and it is an important functional factor that has been studied deeply at present and has multiple physiological activities such as anti-oxidation, sedative nerve, healthy liver and kidney. The preparation methods include chemical synthesis, plant enrichment and microbiological fermentation. The chemical synthesis method has the advantages of short reaction time, high conversion rate and high purity, but some highly corrosive solvents are used in the production process, which has poor safety and is not easy to be controlled, coupled with severe reaction conditions, high cost, large energy consumption and easy production of chemical residues, so it is mostly used in the chemical industry. Although the plant enrichment method is simple in preparation process, it is difficult to obtain high-concentration GABA due to the low content in the plant itself, so it is not suitable for large-scale production. The microbiological fermentation method has the advantages of high efficiency, mild conditions and short cycle time, so it has broad development space. In 1998, Nomura et al. used lactic acid bacteria with high glutamate decarboxylase activity to produce cheese rich in GABA, and the content of GABA in cheese reached 383 mg/kg, then Huang Jun et al. screened and produced GABA with Lactobacillus brevis up to 76.36 g/L from unsterilized raw milk, Li Yali screened Candida lactis producing GABA from raw milk, pickled radish and other samples, but there was no literature record of directly extracting GABA from raw material samples.

Traditional fermented milk contains abundant microbial resources. Sayram Ketteki is a kind of traditional fermented yoghurt formed by local Uygur women in Sayram Town, Baicheng County, Xinjiang Uygur Autonomous Region. It is an elastic set-style fermented milk formed by milking, boiling, cooling and adding a small amount of yogurt left by the last fermentation after 6-8 hours of fermentation at the ambient temperature with local cow's milk as raw material, with a white color such as coagulant fat, mellow taste, sweet and sour refreshing, and sticky texture, and it has the inherent reputation of “brushed yogurt” as nearly 1 m-long silk can be pulled out. Sayram Ketteki contains Lactobacillus helveticus, Kluyveromyces marxianus, Deshi Lactobacillus bulgaricus, Deshi Lactobacillus lactis subspecies, Streptococcus thermophiles, Saccharomyces cerevisiae and other microorganisms. Furthermore, the content of GABA in Sayram Ketteki is much higher than that of similar products. However, if GABA is directly isolated from Sayram Ketteki, on the one hand, the process of isolation and extraction is difficult and the cost is high. On the other hand, the content of GABA in the finished Sayram Ketteki product obtained by traditional fermentation is higher than that of other fermented milk, but as an industrial application, the content thereof needs to be further improved. Therefore, how to ferment Sayram Ketteki to get GABA is a technical problem to be solved urgently in this field.

### SUMMARY OF THE INVENTION

In order to solve the above-mentioned technical problems, the present invention provides a method for producing γ-aminobutyric acid (GABA) by fermentation of native strain from raw material of Sayram Ketteki, and the content of GABA in the transfer solution obtained by this method can be up to 450 μg/mL.

The present invention adopts the following technical solution:

A method for producing γ-aminobutyric acid (GABA) by fermentation of native strain from raw material of Sayram Ketteki, comprising the following steps:

(1) strain isolation: taking cow's milk collected in Sayram Town as the raw material, dissolving the raw material in the normal saline for dilution to obtain diluents with different gradients, coating the diluents with different gradients in solid media for culture respectively, picking up as many colonies with different forms as possible from each plate, and repeatedly performing streaking for numbering till a pure single colony is observed with the naked eye without other miscellaneous colonies, picking up the isolated strains and storing them in a glycerin cryopreservation tube for cryopreservation respectively;

(2) production of GABA by fermentation of the strain: inoculating the preserved strains in liquid medium for culture, wherein the liquid medium is MRS or YPD medium, and inoculating them in improved MRS or YPD medium for culture at 28-32° C. for 22-25 h after activation for twice. That is, a certain strain is isolated from a certain medium, and is activated in the corresponding liquid medium in this step, followed by fermentation transformation in the corresponding modified liquid medium.

As a further improvement of the present invention, the concentration of normal saline described in Step (1) is 0.85 wt %.

As a further improvement of the present invention, the diluent described in Step (1) has a concentration gradient of 10-2, 10-3, 10-4 and 10-5.

As a further improvement of the present invention, the solid media described in Step (1) are MRS and YPD solid media, the diluents with four gradients are coated in MRS and YPD solid media respectively, and cultured at 37° C. and 28° C. for 48 h and 96 h, respectively. Due to the existence of multiple strains in the raw materials, in order to get as many different strains as possible, two media, i.e. MRS and YPD solid media, are used for screening during the isolation.

As a further improvement of the present invention, the concentration of glycerin described in Step (1) is 30 wt %.

As a further improvement of the present invention, the temperature for cryopreservation described in Step (1) is −80° C.

As a further improvement of the present invention, the composition of the improved MRS medium is as follows: 5 parts of peptone, 5 parts of beef extract, 20 parts of yeast powder, 5 parts of glucose, 5 parts of sodium acetate, 0.1 parts of Tween 80, 0.2 parts of trisodium citrate, 0.2 parts of dipotassium phosphate, 0.2 parts of magnesium sulfate, 0.05 parts manganese sulfate, pH6.5, containing 10 g/L sodium glutamate. The composition of the modified YPD medium is as follows: 2 parts of glucose, 2 parts of peptone, 1 part of yeast powder, pH6.0, containing 10g/L sodium glutamate.

The present invention also provides a GABA product obtained by the above-mentioned method, wherein the content of GABA can reach 450 μg/mL.

The present invention has the following technical effects:

Although the content of GABA in traditional Sayram Ketteki is higher than that in common fermented milk, it is difficult to isolate and achieve industrial application output. In the present invention, the strains are isolated from the raw material of Sayram Ketteki—cow's milk in Sayram town first, and then preserved at low temperature for a period of time, activation culture is carried out for twice, the strain transfer solution is finally obtained, the content of GABA in the transfer solution can be up to 450 μg/mL, the content thereof is much higher than that of finished fermented milk product, and much higher than that of fermented milk of common raw materials, which is convenient for industrial production. The fermentation condition control is simple and the energy consumption is low.

### BRIEF DESCRIPTION OF THE FIGURES

FIG. 1 is a standard working curve of γ-aminobutyric acid (GABA);

FIG. 2 is a chromatogram of GABA standard substance; and

FIG. 3 is a chromatogram of the transfer solution in Embodiment 1.

### DETAILED DESCRIPTION OF THE PREFERRED EMBODIMENTS

Multiple exemplary embodiments of the present invention are described in detail. Such detailed description shall not be considered as a limitation of the present invention, but shall be understood as a more detailed description of certain aspects, characteristics and embodiments of the present invention. It shall be understood that the terms described in the present invention are used only to describe particular embodiments and are not intended to limit the present invention.

In addition, the range of values in the present invention shall be understood to mean that each intermediate value between the upper and lower limits of the range is also specifically disclosed. The intermediate value in any stated value or within the stated range and each small range between any other stated values or intermediate values within the range are also included in the present invention. These small ranges of upper and lower limits may be included or excluded independently.

Unless otherwise stated, all technical and scientific terms used herein have the same meaning that would normally be understood by a regular technician in the field described in the present invention. Although the present invention only describes the preferred method and material, any method and material similar or equivalent to those described herein may also be used in the implementation or test of the present invention. All literatures referred to in the Specification are incorporated by citation to disclose and describe methods and/or materials relevant to the same. In the event of conflict with any incorporated literature, the contents of the Specification shall prevail.

A method for producing γ-aminobutyric acid (GABA) by fermentation of native strain from raw material of Sayram Kenai, comprising the following steps:

(1) strain isolation: taking cow's milk collected in Sayram Town as the raw material, dissolving the raw material in the normal saline for dilution to obtain diluents with different gradients, coating the diluents with different gradients in solid media for culture respectively, picking up as many colonies with different forms as possible from each plate, and repeatedly performing streaking for numbering till a pure single colony is observed with the naked eye without other miscellaneous colonies, picking up the isolated strains and storing them in a glycerin cryopreservation tube for cryopreservation respectively;

(2) production of GABA by fermentation of the strain: inoculating the preserved strains in liquid medium for culture, and inoculating them in improved MRS or YPD medium for culture at 28-32° C. for 22-25 h after activation for twice.

As a preferred embodiment, the concentration of normal saline described in Step (1) is 0.85 wt %.

As a preferred embodiment, the diluent described in Step (1) has a concentration gradient of 10-2, 10-3, 10-4 and 10-5.

As a preferred embodiment, the solid media described in Step (1) are MRS and YPD solid media, the diluents with four gradients are coated in MRS and YPD solid media respectively, and cultured at 37° C. and 28° C. for 48 h and 96 h, respectively.

As a preferred embodiment, the concentration of glycerin described in Step (1) is 30 wt %.

As a preferred embodiment, the temperature for cryopreservation described in Step (1) is −80° C.

As a preferred embodiment, the composition of the improved MRS medium is as follows: 5 parts of peptone, 5 parts of beef extract, 20 parts of yeast powder, 5 parts of glucose, 5 parts of sodium acetate, 0.1 parts of Tween 80, 0.2 parts of trisodium citrate, 0.2 parts of dipotassium phosphate, 0.2 parts of magnesium sulfate, 0.05 parts manganese sulfate, pH6.5, containing 10 g/L sodium glutamate. The composition of the modified YPD medium is as follows: 2 parts of glucose, 2 parts of peptone, 1 part of yeast powder, pH6.0, containing 10 g/L sodium glutamate.

The following is detailed description for the technical solution of the present invention through specific embodiments.

Embodiment 1: method for producing γ-aminobutyric acid (GABA) by fermentation of native strain from raw material of Sayram Ketteki

Collect cow's milk from local farmers in Sayram Town, Baicheng County, Xinjiang Uygur Autonomous Region, and take it as the raw material. Transfer 1 mL of stock solution into a 9 mL test tube with 0.85% of normal saline in the clean bench, mix evenly to obtain the diluent with the concentration of 10-1, repeat the above steps in order to dilute for four times to obtain the diluents with the concentration gradient of 10-2, 10-3, 10-4 and 10-5, select the test tubes of diluents with the concentration gradient of 10-2, 10-3, 10-4 and 10-5 to absorb 100 μL of the diluents respectively, and coat the diluents on MRS and YPD solid media for culture at 37° C. and 28° C. for 48 h and 96 h, respectively. Pick up as many colonies with different forms as possible from each plate, and repeatedly perform streaking for numbering till a pure single colony is observed with the naked eye without other miscellaneous colonies, pick up the isolated strains and store them in a glycerin cryopreservation tube with the concentration of 30% for cryopreservation at −80° C. for standby. Inoculate the preserved strains the liquid medium, and culture the strains screened from the solid medium in the corresponding liquid medium at 30° C. for 18h. Inoculate them in the improved MRS or YPD medium according to 4% of the inoculation amount after activation for twice, wherein the composition of the improved MRS medium (g/L) is as follows: 5 parts of peptone, 5 parts of beef extract, 20 parts of yeast powder, 5 parts of glucose, 5 parts of sodium acetate, 0.1 parts of Tween 80, 0.2 parts of trisodium citrate, 0.2 parts of dipotassium phosphate, 0.2 parts of magnesium sulfate, 0.05 parts manganese sulfate, pH6.5, containing 10 g/L sodium glutamate; then culture them at 30° C. for 24 h; the composition of the modified YPD medium (g/L) is as follows: 20 parts of glucose, 20 parts of peptone, 10 part of yeast powder, pH6.0, containing 10 g/L sodium glutamate; then culture them at 30° C. for 48 h to obtain the transfer solution product.

Reference 1:

Collect cow's milk from local artificial cattle farms in Xinxiang City, Henan Province, and take it as the raw material. Transfer 1mL of stock solution into a 9 mL test tube with 0.85% of normal saline in the clean bench, mix evenly to obtain the diluent with the concentration of 10-1, repeat the above steps in order to dilute for four times to obtain the diluents with the concentration gradient of 10-2, 10-3, 10-4 and 10-5, select the test tubes of diluents with the concentration gradient of 10-2, 10-3, 10-4 and 10-5 to absorb 100 μL of the diluents respectively, and coat the diluents on MRS and YPD solid media for culture at 37° C. and 28° C. for 48 h and 96 h, respectively. Pick up as many colonies with different forms as possible from each plate, and repeatedly perform streaking for numbering till a pure single colony is observed with the naked eye without other miscellaneous colonies, pick up the isolated strains and store them in a glycerin cryopreservation tube with the concentration of 30% for cryopreservation at −80° C. for standby. Inoculate the preserved strains the liquid medium for culture at 30° C. for 18 h. Inoculate them in the improved MRS medium according to 4% of the inoculation amount after activation for twice, wherein the composition of the improved MRS medium (g/L) is as follows: 5 parts of peptone, 5 parts of beef extract, 20 parts of yeast powder, 5 parts of glucose, 5 parts of sodium acetate, 0.1 parts of Tween 80, 0.2 parts of trisodium citrate, 0.2 parts of dipotassium phosphate, 0.2 parts of magnesium sulfate, 0.05 parts manganese sulfate, pH6.5, containing 10 g/L sodium glutamate; then culture them at 30° C. for 24 h; the composition of the modified YPD medium (g/L) is as follows: 20 parts of glucose, 20 parts of peptone, 10 part of yeast powder, pH6.0, containing 10 g/L sodium glutamate; then culture them at 30° C. for 48 h to obtain the transfer solution product.

Reference 2: finished Sayram Ketteki product prepared by local fermentation in Sayram Town, Xinjiang Uygur Autonomous Region

Embodiment 2: determination of GABA content in transfer solution product

(I) Drawing a Standard Working Curve

Dilute the sample, and then choose an appropriate gradient, coat it in the solid medium for culture for 2-5 days. Pick up as many colonies with different forms as possible from each plate, and repeatedly perform streaking for numbering till a pure single colony is observed, store the isolated strains in a glycerin cryopreservation tube with the concentration of 30%, perform activization and passage for twice, inoculate the strains into the fermentation medium at the volume fraction of 4%, and then perform stationary culture for 48-96 h.

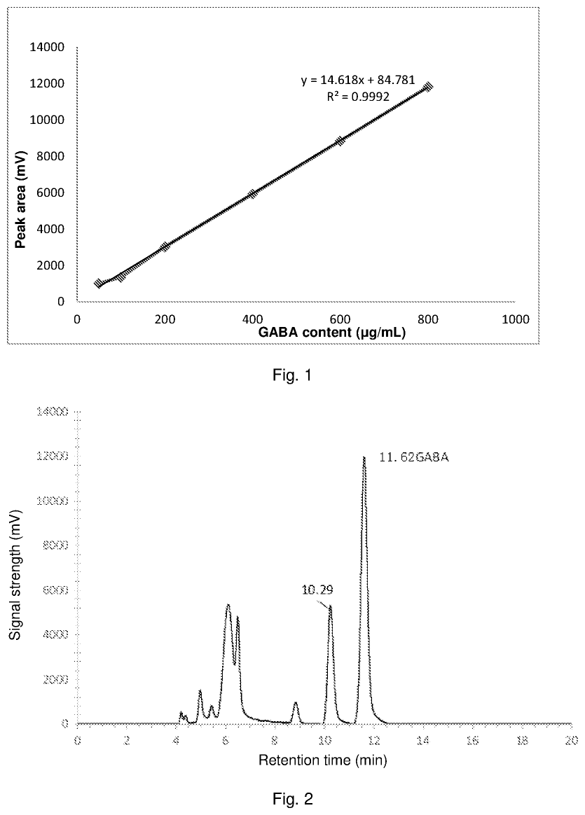

Take 1mL of the transfer solution to centrifuge at 10,000 rpm at 4° C. for 5 min Take 2 82 L of the supernatant with a micro sampling syringe, perform sample application on the silica gel plate (the sample application line is 1.5 cm away from the edge of the chromatographic plate, the sample spacing is 1cm, and the sample spot diameter is no more than 2 mm), and prepare GABA and L-glutamic acid standard samples into 1 g/L standard solution as the control. After the sample spots are dried, place the thin layer of chromatography plate in a closed development tank containing the expanding agent, which is composed of n-butanol alcohol: glacial acetic acid: water=65:15:25 (v: v: v), adding 0.4% of the mass fraction of ninhydrin as the color agent, and do closed ascending development. Take out the chromatography plate for drying and color development at 85° C. for 10 min when the developing agent is about 1 cm away from the front edge of the chromatographic plate. Perform qualitative and quantitative detection for the fermentation product GABA of the screened strains by high performance liquid chromatography, and evaluate the fermentation ability of the obtained strains to produce GAB A. Chromatographic conditions: SHIMADZU-C18 (2) chromatographic column (250×4.6 mm, 5 nm); PDA detector; mobile phase A: methanol, mobile phase B: water (0.05% formic acid); flow rate: 0.8 ml/min; column temperature: 30° C.; detection wavelength: 334nm; sampling method: manual sampling; sample size: 20 μL. Derivation of sample: (1) O-phthalaldehyde (OPA): take 150 mg of OPA to dissolve in 3 mL of methanol first, then dissolve in 27 mL of 0.1 mol/L sodium tetraborate buffer solution, then add in 500 μL of mercaptoethanol, and finally add in 30 mg of ascorbic acid for mixing evenly. (2) Derivation of sample: use a pipette to suck up 700 μL of fermentation supernatant and 7000 μL of OPA derivative reagent successively into a 2 mLEP tube, shake evenly, perform derivation for 2 min, filter with a 0.22 nm filter membrane for 1-2 times, then perform sample injection immediately. Meanwhile, draw a standard curve of GABA: dilute the GABA standard solution (1 mg/mL) into six standard working solutions (50 μg/mL, 100 μg/mL, 200 μg/mL, 400 μg/mL, 600 μg/mL, 800 μg/mL) with double distilled water respectively, perform derivation and sample injection, then take the chromatographic peak area of GABA as the ordinate and the corresponding mass concentration as the abscissa to draw the standard working curve, as shown in FIG. 1. The standard curve equation is y=14.618×+84.781, and the correlation coefficient is R2=0.9992. The GABA standard substance is detected by chromatograph, and the chromatogram thereof is as shown in FIG. 2.

(II) Calculating the Content of GABA

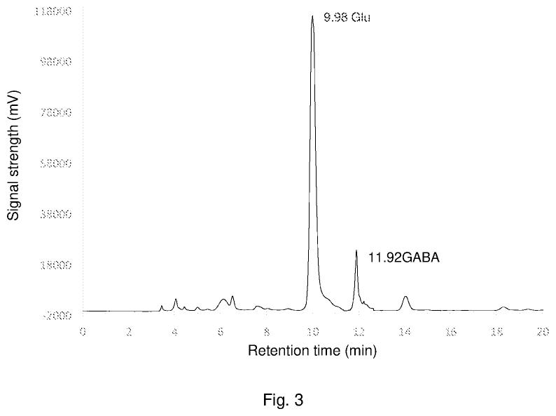

The strain transfer solutions obtained from Embodiment 1 and Reference 1 and the finished yoghurt product obtained from Reference 2 are detected by chromatograph. The chromatogram of Embodiment 1 is as shown in FIG. 3. It can be seen from FIG. 3 that the transfer solution contains GABA. By substituting the peak area into the above-mentioned standard curve equation, it can calculate that the content of GABA in the transfer solution of the strain in Embodiment 1 can reach 450 μg/mL, the content of GABA in the transfer solution of the strain in Reference 1 is 216 μg/mL, and the content of GABA in the finished yogurt product in Reference 2 can reach 409 μg/mL.

Under the condition of not deviating from the scope or spirit of the present invention, multiple improvements and variations may be made to the specific implementation mode of the Specification in the present invention, which is obvious to the technicians in this field. Other implementation modes derived from the Specification of the present invention are obvious to the technicians. The Specification and embodiments of the present invention are illustrative only.

1. A method for producing γ-aminobutyric acid (GABA) by fermentation of native strain from raw material of Sayram Ketteki, comprising the following steps:
(1) strain isolation: taking cow's milk collected in Sayram Town as the raw material, dissolving the raw material in the normal saline for dilution to obtain diluents with different gradients, coating the diluents with different gradients in solid media for culture respectively, picking up as many colonies with different forms as possible from each plate, and repeatedly performing streaking for numbering till a pure single colony is observed with the naked eye without other miscellaneous colonies, picking up the isolated strains and storing them in a glycerin cryopreservation tube for cryopreservation respectively;
(2) production of GABA by fermentation of the strain: inoculating the preserved strains in liquid medium for culture, and inoculating them in improved MRS or YPD medium for culture at 28-32° C. for 22-25 h after activation for twice.

2. The method for producing γ-aminobutyric acid (GABA) by fermentation of native strain from raw material of Sayram Ketteki according to claim 1, wherein the concentration of normal saline described in Step (1) is 0.85 wt %.

3. The method for producing γ-aminobutyric acid (GABA) by fermentation of native strain from raw material of Sayram Ketteki according to claim 1, wherein the diluent described in Step (1) has a concentration gradient of 10-2, 10-3, 10-4 and 10-5.

4. The method for producing γ-aminobutyric acid (GABA) by fermentation of native strain from raw material of Sayram Ketteki according to claim 1, wherein the solid media described in Step (1) are MRS and YPD solid media, the diluents with four gradients are coated in MRS and YPD solid media respectively, and cultured at 37° C. and 28° C. for 48 h and 96 h, respectively.

5. The method for producing γ-aminobutyric acid (GABA) by fermentation of native strain from raw material of Sayram Ketteki according to claim 1, wherein the concentration of glycerin described in Step (1) is 30 wt %.

6. The method for producing γ-aminobutyric acid (GABA) by fermentation of native strain from raw material of Sayram Ketteki according to claim 1, wherein the temperature for cryopreservation described in Step (1) is −80° C.

7. The method for producing γ-aminobutyric acid (GABA) by fermentation of native strain from raw material of Sayram Ketteki according to claim 1, wherein the composition of the improved MRS medium is as follows: 5 parts of peptone, 5 parts of beef extract, 20 parts of yeast powder, 5 parts of glucose, 5 parts of sodium acetate, 0.1 parts of Tween 80, 0.2 parts of trisodium citrate, 0.2 parts of dipotassium phosphate, 0.2 parts of magnesium sulfate, 0.05 parts manganese sulfate, pH6.5, containing 10 g/L sodium glutamate; the composition of the modified YPD medium is as follows: 2 parts of glucose, 2 parts of peptone, 1 part of yeast powder, pH6.0, containing 10 g/L sodium glutamate.

8. A GABA product obtained by the method according to claim 1, wherein the content of GABA can reach 450 μg/mL.
